(19) United States
(12) Patent Application Publication
(10) Pub. No.: US 2009/0246203 A1
(43) Pub. Date: Oct. 1, 2009

(54) METHOD FOR IMMUNOADSORPTION BY MEANS OF AUTOANITGENS

(76) Inventors: Angelika Lüking, Bochum (DE); Christian Scheer, Dortmund (DE); Verena Gruss Trappe, Dortmund (DE); Claudia Gutjahr, Dortmund (DE); Kirsten Schulte, Bochum (DE)

Correspondence Address: CONNOLLY BOVE LODGE & HUTZ, LLP, P O BOX 2207, WILMINGTON, DE, 19899, US

(21) Appl. No.: 12/162,240

(22) PCT Filed: Jan. 24, 2007
(86) PCT No.: PCT/DE07/00139

§ 371 Date: Jan. 9, 2009

(30) Foreign Application Priority Data
Jan. 25, 2006 (DE) 10-2006-003-782.0

(51) Int. Cl. A61K 39/395; C40B 30/04; A61P 37/06

(52) U.S. Cl. 424/140.1; 506/9

(57) ABSTRACT

The invention relates to a novel method for immunoadsorption therapy of human autoimmune diseases by means of immobilized or fixed autoantigens.

The invention concerns a novel method for immunoadsorption by human or mammal autoimmune diseases. Most commonly, autoimmune diseases exhibit indication-specific antibodies (von Muehlen, C A., Tan E. M. (1995) Autoantibodies in the diagnosis of systemic rheumatic diseases. Semin. Arthrithis Rheum. 24, 323-358). 5% of the 10 world population is affected by autoimmune diseases (Davidson, A, Diamond B. (2001) Autoimmune diseases. N. Engl. J. Med. 354, 340-350).

Such antibodies, e.g. against cardiac structures, were indicated in recent years for Dilatative Cardiomyopathy (DCM). This is attributed in patients to “cardiotoxic antibodies”, which are directed against the heart muscle cells. These autoantibodies could be eliminated by immunoadsorption therapy (Doerffel W V, Felix S B, Wallukat G., et al. Short-term hemodynamic effects of immunoadsorption in dilated cardiomyopathy. Circulation 1997; 95(8):1994-1997). With immunoadsorption therapy it concerns a special dialysis, which targets ridding the blood of autoantibodies. DCM patients were treated with the help of immunoadsorption therapy in monthly intervals over three months and the hemodynamics were analyzed with the help of Swan Ganz monitoring. Increases in the heart index, the beat volume index and the left ventricular ejection fraction (echocardiography) could be proved. At the same time a decrease in vascular system resistance was recorded.

Immunoadsorption is a special method of adsorption, which serves immunological trait; it is state of the art technology already introduced for the treatment of patients with autoimmune diseases. With this therapy, in patients with autoimmune diseases harmful substances can be efficiently and partly in large quantities removed from the body. As a result, often shortly after the start of treatment a rapid improvement of the symptomatology can be achieved. Subsequently however, adverse temporary or long-term remissions are observed.

Examples of successful therapies are Rheumatoid Arthritis (RA) and Idiopathic Thrombocytopenic Purpura (ITP). RA and ITP are both autoimmune diseases, which are distinguished by the occurrence of autoantibodies in high titers and/or serum concentrations, these autoantibodies are directly involved in the formation of tissue damage and the progression of the disease.

As state of the art technology for immunoadsorption therapy adsorber materials, e.g. in the appropriate columns or membrane modules, were used with immobilized Protein A or Protein G.

Protein A or Protein G (for example, obtained from Miltenyi Biotec, Germany) are components of the cell wall of the bacterium Staphylococcus and have the capacity to bind non-selective immunoglobulins of the IgG class because of their high affinity to the Fc portion of the IgG antibodies (Class 1, 2 and 4). With less affinity they likewise bind certain IgG3, IgA and IgM antibodies.

In state of the art with the help of an adsorption column all IgG are removed from the body of the patient, under which likewise the harmful circulation immune complexes are found with RA and immunoglobulins against thrombocytes for IPT.

In order to prevent weakening of the immune system, intact human immunoglobulins must be traced back in a time consuming manner to the patient. This substitution is not free of side effects. Such an adsorption column has been approved in the USA since 1987 for the treatment of ITP and is in use.

More than 11,000 patients were treated here.

Also in other rheumatological indications such as Systemic Lupus Erythematosis (SLE) or Wegener's Granulomatosis, good treatment prospects exist.

Likewise, immunoadsorption therapy for patients with a severe course of SLE is known. Immunoadsorption was performed with immunoglobulin-binding substrates in patients with active SLE, for whom a cyclophosphamide therapy is not sufficient or appeared to be contraindicated.

Separated patient plasma was adsorbed with polyclonal sheep antibodies against human immunoglobulin (Ig Therasorb columns), after respectively three immunoglobulin aphereses were substituted with 15 g each of immunoglobulin from healthy donors.

The immunoadsorption is a replacement for the conventional plasma pheresis and appears with SLE with a difficult disease course as an important additional opportunity for immunosuppressive therapy.

Likewise, the immunoadsorption therapy uses, as described in Artificial Organs, (1996), 986-990, the amino acids tryptophan or phenylalanin to bind the PVA portions and is therefore inconvenient and costly. Furthermore, with this therapy also substances, which should not be removed from the plasma, such as IgG and IgM, are separated from the plasma in comparable volumes.

In state of the art technology it is detrimental that the adsorption within the context of immunoadsorption therapy (and/or dialysis) is unspecific and other valuable blood components are removed along with the indication-specific and relevant autoantibodies, which again must be costly for the patient. This carries significant risks for long-term use.

Therefore, the object of this invention is to ensure specific protein-protein interactions, in a manner so that the autoantibodies can be selectively removed.

The solution of this object is created by the preparation of autoantigens immobilized or fixed in the adsorbent.

Therefore, the invention concerns a therapeutic method for treatment and prophylaxis of autoimmune diseases, where adsorption of autoantibodies is derived from blood or blood plasma extracorporeally by means of autoantigens (hereinafter called “the invention therapy”).

The invention concerns likewise a method for the manufacture of an adsorbent for adsorption of antibodies in blood or blood plasma in a preferred embodiment, comprising the following steps:
a.) Provision of support materials
b.) Immobilization or fixing of at least one autoantigen in this support material and bringing the adsorbent into contact with blood or blood plasma (hereinafter referred to as “invention methods”).

In a preferred embodiment the method in accordance with the invention is carried out extracorporeally. In addition, the blood or blood plasma can continuously flow along in another embodiment in the support material containing autoantigens.

“Extracorporeal” in the sense of this invention means that the invention therapy or the invention method is performed outside of the human body (synonym: “ex vivo”). The invention therapy is carried out for the treatment or prophylaxis of autoimmune diseases. Autoimmune diseases are those that essentially exhibit indication-specific autoantibodies that are potentially capable of damaging the body in any way, in particular attacking the body's tissues (e.g. due to overactive T-cells).

For the purposes of this invention the autoimmune diseases include non-exclusively the following:
Alopecia Areata
Anemia, cold autoimmune-hemolytic (AIHA) (Cold agglutinin disease)
Anemia, warm autoimmune-hemolytic (AIHA)
Anemia, autoimmune-hemolytic Donath-Landsteiner (paroxysmal cold hemoglobinuria)
Anemia, pernicious (Biermer's Disease, Addison-Anemia)
Antiphospholipid Syndrome (APS)
Temporal arteritis (Giant cell arteritis)
Atherosclerosis (Arteriosclerosis, Artery calcification)
Autoimmune-Adrenalitis (autoimmune adrenal gland atrophy, Addison's Disease)
Chronic Fatigue Syndrome (Chronic Fatigue Immune Dysfunction Syndrome; CFIDS)
Chronic-Inflammatory, Demyelinating Polyneuropathy (CIDP)
Churg-Strauss Syndrome
Cogan Syndrome
Ulcerative Colitis
CREST-Syndrome (Syndrome with Calcinosis cutis, Raynaud's phenomenon, motility disorders of the esophagus Sclerodactylia and Teleangiectasia)
Diabetes mellitus Type 1 (Insulin-dependent Diabetes mellitus)
Dilatative Cardiomyopathy (DCM)
Dermatitis Herpetiformis During
Dermatomyositis
Fibromyalgia
Gastritis, chronic autoimmune
Goodpasture's Syndrome (Anti-GBM mediated Glomerulonephritis)
Guillain-Barre-Syndrome (GBS; Polyradiculoneuritis)
Hashimoto Thyroiditis
Hepatitis, autoimmune (Chronic active Hepatitis; CAH)
Idiopathic Pulmonary Fibrosis (IPF)
Immune-thrombocytopenic Purpura (Werlhof's Disease; ITP)
Infertility, autoimmune
Inner Ear Disease, autoimmune (AIED)
Juvenile Rheumatoid Arthritis (Still's Disease, Still Syndrome)
Cardiomyopathy, autoimmune
Cold Agglutinin Disease (see also: Anemia, cold autoimmune-hemolytic)
Cryoglobulinemia, essentially mixed
Lambert-Eaton-Syndrome
Lichen sclerosus
Lupus erythematosus (discoid Form)
Lyme-Arthritis (Borrelia Arthritis, Lyme Disease)
Mixed collagenosis (Mixed Connective Tissue Disease; MCTD)
Addison's Disease (see also: Autoimmune Adrenalitis; autoimmune Adrenal Gland Atrophy)
Basedow's Disease (Graves Disease)
Behcet's Disease
Crohn's Disease
Bechterew's Disease (Spondylitis ankylosans)
Meniere's Disease (Meniere's Disease)
Reiter's Disease (see also: Reiter Syndrome)
Multiple Sclerosis (MS; Encephalomyelitis disseminata; Charcot's Disease)
Myasthenia gravis (Myasthenia; MG)
Ophthalmia, sympathetic
Pemphigoid, cicatricial
Pemphigoid, bullous
Pemphigus vulgaris
Pernicious Anemia (Biermer's Disease; Addison-Anemia; see also Anemia, pernicious)
Polyarteritis nodosa (Periarteritis nodosa)
Polychondritis (Panchondritis)
Polyglandular Autoimmune-(PGA)-Syndrome (Schmidt's Syndrome)
Polymyalgia rheumatica
Polymyositis
Polyneuropathy, Chronic-Inflammatory, Demyelinating (see also: Chronic-Inflammatory, Demyelinating Polyneuropathy, CIDP)
Primari biliary Cirrhosis (PBC; primary Autoimmune-Cholangitis)
Psoriasis (Psoriasis)
Reiter's Syndrome (Reiter's Disease; Urethro-Conjunctival-Synovial Syndrome)
Rheumatic Fever
Great cell arteritis (see also: Temporal arteritis)
Rheumatoid Arthritis (chronic Polyarthritis, “articular rheumatism”)
Sarcoidosis (Boeck's Disease, Besnier-Boeck-Schaumann's Disease)
Sjögren-Syndrome
Scleroderma
Spondylitis ankylosans (see also: Bechterew's Disease)
Sprue/Celiac
Stiff-Man-Syndrome (SMS; Moersch-Woltmann-Syndrome)
Systemic Lupus erythematosus (SLE)
Takayasu Arteritis (Takayasu Disease; Aortic Arch Syndrome)
Transient Gluten Intolerance
Uveitis, autoimmune
Vasculitis
Vitiligo (Vitiligo)
Wegener's Granulomatosis (see: Wegener's Disease)

Furthermore, the invention therapy or the invention method is applicable in the case of transplant rejection, such as the transplant of various organs (HLA, hyperimmunization), Acute Vascular Rejection (AVR).

Preferred is the treatment of such autoinmune diseases by means of the invention therapy or the treatment method, which exhibits an elevated titer and/or serum concentration of one or multiple antibodies, such as Rheumatoid Arthritis (RA) and Idiopathic Thrombocytopenic Purpura (ITP).

Another subject of this invention concerns the identification of the appropriate autoantigens for selective removal of the autoantibodies.

The autoantigens according to the invention can be identified primarily by means of protein micro- and macroarrays.

In the context of this invention, the term protein micro- and macroarrays covers any arrangement of proteins on a surface of a solid carrier. In the context of this invention “Array” is a synonym for “arrangement” and provided this “array” is used for the identification and characterization of proteins, in particular autoantigens, hereunder understood as an “assay” or “biochip.”

For this purpose, such a micro- or macro-“array” is incubated with patient serums. In a preferred embodiment the arrangement of this type is designed so that the proteins represented in the arrangement are present in the form of a grid. Furthermore, such arrangements are preferred that allow a high-density (high-density) arrangement of the proteins. Such high-density arrangements are revealed, for example, in the WO 99/57311 and WO 99/57312.

The term “solid carrier” includes items such as a filter, a membrane, magnetic beads, a silicon wafer, glass, metal, a chip, a massspectrometric target or a matrix. PVDF or nylon are preferred as a filter (e.g. Hybond N+, GE Health Care). Another preferred embodiment for the invention arrangement includes a grid that occupies the magnitude of a microtiter plate (96 Wells, 384 Wells or more), a silicon wafer, a chip, a massspectrometric target or a matrix.

Another preferred embodiment for the invention arrangement is a grid that occupies the magnitude of a microtiter plate (96 Wells, 384 Wells or more), a silicon wafer, a chip, a massspectrometric target, or a matrix.

Protein micro- and macroarrays allow the rapid and high parallel detection of a variety of specific binding analysis molecules in a single experiment. For the manufacture of protein micro- and macroarrays it is necessary to have available the proteins required. For this reason protein expression libraries have been established. The high performance cloning of a defined open reading frame is a possibility (Heyman, J. A., Cornthwaite, J., Foncerrada, L., Gilmore, J. R., Gontang, E., Hartman, K. J., Hernandez, C. L., Hood, R., Hull, H. M., Lee, W. Y., Marcil, R., Marsh, E. J., Mudd, K. M., Patino, M. J., Purcell, T. J., Rowland, J. J., Sindici, M. L. and Hoeffler, J. P. (1999) Genome-scale cloning and expression of individual open reading frames using topoisomerase I-mediated ligation. Genome Res, 9, 383-392; Kersten, B., Feilner, T., Kramer, A., Wehrmeyer, S., Possling, A., Witt, I., Zanor, M. I., Stracke, R., Lueking, A., Kreutzberger, J., Lehrach, H. and Cahill, D. J. (2003) Generation of Arabidopsis protein chip for antibody and serum screening. Plant Molecular Biology, 52, 999-1010; Reboul, J., Vaglio, P., Rual, J. F., Lamesch, P., Martinez, M., Armstrong, C M., Li, S., Jacotot, L., Bertin, N., Janky, R., Moore, T., Hudson, J. R., Jr., Hartley, J. L., Brasch, M. A., Vandenhaute, J., Boulton, S., Endress, G. A., Jenna, S., Chevet, E., Papasotiropoulos, V., Tolias, P. P., Ptacek, J., Snyder, M., Huang, R., Chance, M. R., Lee, H., Doucette-Stamm, L., Hill, D. E. and Vidal, M. (2003) C. elegans ORFeome version 1.1: experimental verification of the genome annotation and resource for proteome-scale protein expression. Nat Genet, 34, 35-41.; Walhout, A. J., Temple, G. F., Brasch, M. A., Hartley, J. L., Lorson, M. A., van den Heuvel, S. and Vidal, M. (2000) GATEWAY recombinational cloning: application to the cloning of large numbers of open reading frames or ORFeomes. Methods Enzymol, 328, 575-592). However, such an approach is strongly connected with the progress of the genome sequencing projects, their quality and the annotation of the gene sequences. In addition, the determination of the expressed sequence is not always clear due to differential splice procedures.

This problem can be bypassed through the use of cDNA expression libraries (Buessow, K., Cahill, D., Nietfeld, W., Bancroft, D., Scherzinger, E., Lehrach, H. and Walter, G. (1998) A method for global protein expression and antibody screening on high-density filters of an arrayed cDNA library. Nucleic Acids Research, 26, 5007-5008; Buessow, K., Nordhoff, E., Luebbert, C, Lehrach, H. and Walter, G. (2000) A human cDNA library for high-throughput protein expression screening. Genomics, 65, 1-8; Holz, C, Lueking, A., Bovekamp, L., Gutjahr, C, Bolotina, N., Lehrach, H. and Cahill, D. J. (2001) A human cDNA expression library in yeast enriched for open reading frames. Genome Res, 11, 1730-1735; Lueking, A., Holz, C, Gotthold, C, Lehrach, H. and Cahill, D. (2000) A system for dual protein expression in Pichia pastoris and Escherichia coli, Protein Expr. Purif., 20, 372-378). Here the cDNA of a particular tissue will be cloned into a bacterial or a yeast expression vector or other appropriate vector. The vectors used for the expression are distinguished in general thus by the fact that they carry inducible promoters, with which the time of the protein expression can be controlled. In addition, some expression vectors exhibit sequences for the so-called affinity epitopes or proteins, which on the one hand permit the specific detection of recombinant fusion-proteins by means of antibodies directed against the affinity epitope, and on the other hand enable the specific purification by affinity chromatography (IMAC). Furthermore, some of these expression vectors contain preferable inducible promoters. The induction of the expression can be carried out e.g. by means of an inductor such as IPTG. Appropriate expression vectors are described in Terpe et al. (Terpe T Appl Microbiol Biotechnol. 2003 Jan; 60(5):523-33). In addition, the expression product is present preferably in the form of a fusion protein, which, for example, contains at least one affinity epitope or “Tag”. The Tag can contain c-myc, His-Tag, Arg-tag, FLAG, alkaline phosphatase, V5-Tag, T7-Tag or Strep-Tag, HAT-tag, NusA, S-tag, SBP-tag, Thioredoxin, DsbA, a fusion protein, preferably a cellulose-binding domain, green fluorescent protein, maltose binding protein, calmodulin-binding protein, glutathione s-transferase or lacZ. For example, the gene products of a cDNA expression library from human fetal brain tissue in the bacterial expression system Escherichia coli were successfully arranged in high-density format on a membrane and could be successfully screened with various antibodies regarding specific protein antibody interaction. It was shown that the proportion of full-length proteins was at least 66%. The recombinant proteins of this library could furthermore be expressed and purified Braun P., Hu, Y., Shen, B., Halleck, A., Koundinya, M., Harlow, E. and LaBaer, J. (2002) Proteome-scale purification of human proteins from bacteria. Proc Natl Acad Sei U S A, 99, 2654-2659; Buessow (2000) supra; Lueking, A., Horn, M., Eickhoff, H., Buessow, K., Lehrach, H. and Walter, G. (1999) Protein microarrays for gene expression and antibody screening. Analytical Biochemistry, 270, 103-111. Such protein micro- and macroarrays on the basis of cDNA expression libraries are, in particular, a subject of WO 99/57311 and WO 99/57312. Expression libraries are known to the expert, these may be such published standard words as Sambrook et al, “Molecular Cloning, A laboratory handbook, 2nd edition (1989), CSH press, Cold Spring Harbor, New York. Furthermore, expression libraries are preferable that are tissue-specific (e.g. human tissue, in particular human organs). Moreover, such expression libraries are likewise in accordance with the invention that can be maintained by means of exon-trapping. Instead, the expression library can be called a synonym of an expression bank. Furthermore, protein micro- and macroarrays or corresponding expression libraries are preferable, that exhibit no redundancy (so called: Uniclone®-Biochips) and can be produced in accordance with the tenets of the WO 99/57311 and WO 99/57312. These preferred Uniclone-Biochips display a high percentage of non-defective completely expressed proteins of a cDNA expression library (see example). In particular, it is possible to produced protein micro- and macroarrays that represent proteins from disease-specific tissue of autoimmune diseases (Lueking (2003) supra, Lueking A., Huber O., Wirths C, Schulte K., Stieler K. M., Blume-Peytavi ü., Kowald A., Hensel-Wiegel K, Tauber R., Lehrach H, Meyer, H. E., Cahill J. D, Profiling of Alopecia Areata Autoantigens Based on Protein Microarray Technology, Molecular & Cellular Proteomics 4: 1382-1390, 2005).

With the help of such protein micro- and macroarrays autoantigens can be clearly identified (Screening), cultivated and purified (protein production) and further characterized. For this purpose, serums, blood or blood plasma were given by the patients over a protein micro- and macroarray (See examples). In particular, in accordance with the invention, by means of a macroarray (e.g. from a human cDNA expression bank with more than 10,000 clones) potential autoantigens are identified, which based on a microarray (selection of potential clone) are confirmed by means of patient serums (containing autoantibodies).

Thus, the invention also concerns a method for the identification and characterization of at least one autoantigen, consisting of the following steps:
a.) Incubation of a first array containing proteins with patient serums, blood, plasma,
b.) Identification of the proteins with autoantigen autoantibody interactions and
c.) Re-Arraying of the identified proteins in a second array,
d.) Incubation of the array from c.) with patient serums, blood, plasma.

In a preferred embodiment the first array of a cDNA expression library (described, supra) exists with at least 10,000 clones or proteins. Furthermore, it is preferable that the first array in a) displays redundant clones or proteins. “Re-Arraying” means that the identified proteins or clones were rearranged in a second array.

The visualization of such autoantibody-autoantigen interactions can be performed by means of fluorescence marking, biotinylation or radioisotope marking in the usual manner. Reading is performed, e.g. by means of a microarray laser scanner.

Understandably, likewise other analytical methods are possible for the identification and characterization of autoantigens (ELISA, BIAcore, et al). The correspondingly identified autoantigens can be isolated, concentrated or cloned by the usual methods.

Thus, the invention therapy concerns those autoantigens that are received from a screening method by means of arbitrary analytical methods, in particular those such as protein micro- and macroarrays, ELISA and/or BIAcore.

Furthermore, the invention therapy concerns those support materials (adsorber), which are capable of fixing or immobilizing autoantigens. In a preferred embodiment the adsorber is biocompatible (Jayabalan M. Sterilization and reprocessing of materials and medical devices—reusability. J Biomater Appl. 1995 Jul; 10(1): 97-112. Review., Ota K. Biocompatibility and capability of hemopurification systems: review and future development. Nephrol Dial Transplant. 1991;6 Suppl 2:86-90. Review). The appropriate support materials (in the broadest sense—a matrix) are those not excluding for example glass, carbohydrates and modified carbohydrates, sepharose, proteins such as collagen or gelatin, silica or organic matrices, polymers such as polyamides, PVDF, nylon, nitrocellulose among others.

The support material (matrix) can be present in the form of spherical, unaggregated particles, so-called beads, fibers or a membrane, where the porosity of the matrix of the surface is increased. The porosity can be achieved for example in the usual manner by the addition of pore builders like Cyclohexanol or 1-Dodecanol to the reaction mixture of the suspension polymerization.

The autoantigens can be introduced to the support material in a known and arbitrary manner. In a preferred embodiment the autoantigen is for example a fusion protein (e.g. supra), which is suitable for adhering to the support material.

In another embodiment of the invention, individual autoantigens can be identified by means of the protein micro- and macroarray provided for the patient and can be introduced into the therapeutic methods. As already only 50-40% or just 20-10% of the autoantigens of an autoimmune disease have been qualitatively identified and specified, a considerable or significant therapy success can already be introduced. For example, for MS 20 autoantigens are known and active. Therefore, already 8 and fewer can lead to a therapeutic success.

In another embodiment, likewise non-individual autoantigens can be used to get an average (5 patients would be considered significant) for the population of patients.

Therefore, the invention also concerns a method where non-individual indication-specific autoantigens of a certain number of patients can be introduced to the invention therapy of an arbitrary population (always greater than the designated specified number) of patients.

Furthermore, the invention concerns a device or kit consisting of support material containing selected autoantigens of all the usual medium, in particular components for the performance of the invention therapy and/or dialysis/immunoadsorption therapy as well as the invention method. The corresponding known devices for dialysis or immunoadsorption therapy can be adapted respectively.

In addition, the invention concerns the use of support material containing autoantigens for immunoadsorption therapy. Furthermore, the invention concerns the use of support material containing autoantigens for adsorption of autoantibodies from the blood or blood plasma of patients with autoimmune diseases.

To prevent unwanted substances, such as substances that originate from the support material, from returning to the bloodstream of the patients with the treated blood and/or blood plasma, if necessary preferably a filter will be located at the outlet of the support material. Therefore, it concerns preferably a particle filter of suitable size.

The following examples serve to further explain the invention without limiting the invention to these.

### EXAMPLES

### Example 1

### High-Throughput Identification of Protein Antigens

For the firm identification of mammalian autoantigens as many of the specific expression products (proteins) as possible must be available in a test format, e.g. in array arrangement, for each respective genome of an organism. This, e.g. in the form a of macroarray on a support material of a suitable size providing protein diversity, was used to identify through incubation with mammalian serum the antibodies contained therein. Hence, it is particularly beneficial if the macroarray provided displays expression clones and/or expression products of a certain amount of redundancy, e.g. between 1-10. Screenings were performed using high-density filters of expression libraries. These high-density filters contain a large protein accumulation with at least 10,000 different proteins. Furthermore, this screening approach was already described as successful (Gutjahr, C., et al., Mouse protein arrays from a T(H) 1 cell cDNA library for antibody screening and serum profiling. Genomics, 2005. 85(3): p. 285-96). For this screening approach a high-density filter of the “human fetal brain” expression library (Buessow, K., (1998), supra), was used as well as the T-lymphocyte specific expression library.

### Example 2

### Validation of the Autoantigens Identified in Example 1 With the Help of Protein Microarrays.

The autoantigen expressed expression clones identified in Example 1 were re-arrayed (i.e positively identified clones were introduced into a second array), cultivated and the expression products were purified by IMAC. For this purpose, the purified proteins were initially checked by SDS gelectrophoresis for their purity and additionally were analyzed massspectrometrically. The purified and characterized proteins of this type were subsequently spotted on a suitable carrier for production of a microarray.

The proteins produced in Example 1 were transferred with a spotting robot to nitrocellulose covered carrier surfaces. Furthermore, the protein biochip received additional control proteins. For process control human IgG and mouse IgG were used. These immunoglobulins were connected by the secondary antibodies necessary for the detection of human autoantibodies and could also used along with the process control for normalization (“inter chip normalization”). The process control proteins were moreover distributed to multiple quadrants of the chip. This allowed a so-called “Intrafield” analysis with which the homogenicity of the surface and/or incubation reaction was checked. With the appropriate bioinformation tools (reviewed in: Hamacher, Michael/Marcus, Katrin/Stuehler, Kai/van Hall, Andre/Warscheid, Bettina/Meyer, Helmut E. (Hrsq.), Proteomics in Drug Research, Methods and Principles in Medicinal Chemistry (Band 28) published by Mannhold, Raimund/Kubinyi, Hugo/Folkers, Gerd) based on the process control protein data the normalization was performed within a screen, as well as the intrafield analysis and if necessary the following comparison within a chip.

Furthermore, the natural autoantigens (NAAs) such as Stathmin or Tubulin, which are found in almost 90% of all serums (independent of whether sick or healthy), are introduced into the array. Thus, for serum screening applications the quality of the serums was controlled. In addition, non-epitope-tagged, non-human proteins such as BSA and Lysozyme were introduced into the protein biochip. These were supplied for determination of the reference value of the background. All these available proteins were spotted in duplicate (and partly in multiple quadrants) on the protein biochip. The protein biochips produced in Example 2 were introduced in Example 5.

### Example 3

### Patient Selection and Serum Collection, Quality Control of the Serum

Patient selection was performed with consideration of the age and sex of the patient (comparability with the control group), as well as the described parameters (clinical and laboratory values) of the course of the illness. Serum collection was performed according to a standardized protocol (taking, centrifuging, aliquoting, shock freezing and storage at −80° C.), where a total of 15 ml of whole blood was taken for serum extraction. A total of more than one hundred samples were characterized and examined collectively for each patient (RA patients)and control group. The sample data (sample and patient IDs, date taken, medication administered) was placed on a standardized sample card when the sample was taken the corresponding samples were collected.

### Example 4

### Database Generation

A database was designed to deal with a high number of clones. Subsequently, this model was implemented and a database-based client software was created. The data of each clone of a collection was included and archived in this database. For example, the sequencing information, the gene ontology classification of a corresponding protein product, the expression rate and the information on quality control such as ID gel and massspectrometric results were stored made accessible through various filters and search functions. A snapshot of the Uniclone® collection of existing knowledge on RA and MS was created. The contents of the database form the basis for section from the population and serve to illustrate the past information for the indications (RA, MS0 in this selection. In addition, interfaces were developed for the existing available applications and new types of applications, which were used for the evaluation of the screening results. At the same time, in particular, special aspects of the chip layout (intrafield analysis), other protein-specific aspects and the partly0automated quality control of the data of the protein biochips were considered.

### Example 5

### Test of the Serum on the Screening Tools

Subsequently, RA and MS patient serums were catalogued and the corresponding data was stored in the appropriate format in the database. The patient serums were then examined with the various screening tools. Proteins (Example 1) were first screened with the use of high-density filters of expression libraries. These high-density filters contain a large protein collection with a least 10,000 different proteins. Furthermore, this screening approach was already described as successful (Gutjahr, C., et al., Mouse protein arrays from a T(H) 1 cell cDNA library for antibody screening and serum profiling. Genomics, 2005. 85(3): p. 285-96). The proteins identified in this screening were introduced into the collection of 2000 and/or 4000 proteins. For this screening approach high-density filters of the “human fetal brain” expression library (Buessow, K., et al., A method for global protein expression and antibody screening on high-density filters of an arrayed cDNA library. Nucleic Acids Research, 1998. 26(21): p. 5007-5008) were used as well as the T-lymphocyte specific expression library.

Serums from approximately 15-20 RA and MS reference patients were examined on the high-density filters. The received data sets were compared with already existing data sets of healthy and/or unremarkable persons.

In the following analysis packets 50-100 RA patients were examined and compared with adults of the “healthy-control group.”

All test data, such as chip production data (production batch, protein batch), patient sample data, as well as the applicable raw data was transferred for image evaluation in the database (Example 4). After image analysis, for all analysis packets the bioinformational evaluation was performed with consideration of the correlation of the patient data with the screening data (Example 6). This bioinformational examination resulted in the selection of putatively relevant protein antigens. The corresponding expression clones of these protein antigens were, as described in Example 8, back-validated.

### Example 6

### Evaluation of the Data and Selection of the Relevant Protein Antigens

After the image analysis the pre-processing of the data (normalization) was performed in order to make the various experiments and batches comparable. In so doing, for a first test set the various normalization methods and statistical tests, which were described for the section of DNA microarrays, were checked regarding their suitability for protein microarrays.

The putative biomarkers determined by these analyses were then examined in Example 5 again with various test serums from patients with other autoimmune diseases such as Sjogren's Syndrome, SLE or Alopecia areata.

### Example 7

Comparative Proteom Study of Biopsy Material from RA Patients for Validation of the Identified Antigens

With the assistance of proprietary technologies, in particular the high-resolution 2D Large Gel Technology (LGT) the Proteome of biopsy materials from 15 RA patients and 15 control subjects was depicted and compared. Differential proteins from the proteome comparison of healthy and RA patients were subsequently statistically evaluated and identified by means of the usual massspectrometry techniques.

In order to increase the statistical relevance of the analysis results, to improve the gel-to-gel comparability and in addition to cover a large dynamic range of singles components within the complex analysis mixture, the proteomes were depicted and compared with the assistance of the state-of-the-art technology 2-D DIGE fluorescence difference gel electrophoresis.

The comparison of the biopsy materials of the control subjects with RA patients was then introduced to confirm the various antigens found in the serum screenings and at the same time to identify any other possible proteins, which are changed significantly in their volume with RA.

### Example 8

### Validation of the Relevant Protein Antigens on the Western Immunoblot

The back-validation of selected protein antigens was performed with another aliquote of the same serum by means of the Western Immunoblot. For this purpose, the corresponding antigens were produced on an analytical scale.

### Example 9

All of the positively validated protein antigens were used for the creation of prototypes. All proteins were purified and examined via SDS-PAGE, as well as MALDI-MS (-MS) for their identity and quality. In previous tests the necessary concentration range for each of the selected protein antigens was determined with the use of a defined test plasma. The isolated autoantigens were introduced onto the usual commercial adsorption column. The patient blood or plasma samples were washed over these columns. Subsequently, it was examined by means of protein biochips and ELISA whether the disease-specific antigens could still be seen in the washed blood or plasma samples.

1. A method for the treatment and prophylaxis of autoimmune diseases, comprising:
adsorbing autoantibodies from blood or blood plasma extracorporeally by means of autoantigens.

2. A method for the manufacture of an adsorbent for adsorption of autoantibodies in blood or blood plasma, comprising:
a.) providing support materials,
b.) immobilizing or fixing of at least one autoantigen to the support material and bringing the adsorbent into contact with the blood or blood plasma

3. A method according to claim 1, characterized in that autoimmune diseases are selected from the group:
Alopecia Areata
Anemia, cold autoimmune-hemolytic (AIHA) (Cold agglutinin disease)
Anemia, warm autoimmune-hemolytic (AIHA)
Anemia, autoimmune-hemolytic Donath-Landsteiner (paroxysmal cold hemoglobinuria)
Anemia, pernicious (Bierrner's Disease, Addison-Anemia)
Antiphospholipid Syndrome (APS)
Temporal arteritis (Giant cell arteritis)
Atherosclerosis (Arteriosclerosis, Artery calcification)
Autoimmune-Adrenalitis (autoimmune adrenal gland atrophy, Addison's Disease)
Chronic Fatigue Syndrome (Chronic Fatigue Immune Dysfunction Syndrome, CFIDS)
Chronic-Inflammatory, Demyelinating Polyneuropathy (CIDP)
Churg-Strauss Syndrome
Cogan Syndrome
Ulcerative Colitis
CREST-Syndrome (Syndrome with Calcinosis cutis, Raynaud's phenomenon, motility disorders of the esophagus Sclerodactylia and Teleangiectasia)
Diabetes mellitus Type I (Insulin-dependent Diabetes mellitus)
Dilatative Cardiomyopathy (DCM)
Dermatitis Herpetiformis During
Dermatomyositis
Fibromyalgia
Gastritis, chronic autoimmune
Goodpasture's Syndrome (Anti-GBM mediated Glomerulonephritis)
Guillain-Barre-Syndrome (GBS; Polyradiculoneuritis)
Hashimoto Thyroiditis
Hepatitis, autoimmune (Chronic active Hepatitis; CAH)
Idiopathic Pulmonary Fibrosis (IPF)
Immune-thrombocytopenic Purpura (Werlhof's Disease; ITP)
Infertility, autoimmune
Inner Ear Disease, autoimmune (AIED)
Juvenile Rheumatoid Arthritis (Still's Disease, Still Syndrome)
Cardiomyopathy, autoimmune
Cold Agglutinin Disease (see also: Anemia, cold autoimmune-hemolytic)
Cryoglobulinemia, essentially mixed
Lambert-Eaton-Syndrome
Lichen sclerosus
Lupus erythematosus (discoid Form)
Lyme-Arthritis (Borrelia Arthritis, Lyme Disease)
Mixed collagenosis (Mixed Connective Tissue Disease; MCTD)
Addison's Disease (see also: Autoimmune Adrenalitis; autoimmune Adrenal Gland Atrophy)
Basedow's Disease (Graves Disease)
Behcet's Disease
Crohn's Disease

9. The use of a support material according to claim 8 containing autoantigens for immunoadsorption therapy.

10. The use of a support material according to claim 8 containing autoantigens for adsorption of autoantibodies from blood or blood plasma.

11. A device of kit consisting of support material containing autoantigens for performance of methods according to claim 1.
Bechterew's Disease (Spondylitis ankylosans)
Meniere's Disease (Meniere's Disease)
Reiter's Disease (see also: Reiter Syndrome)
Multiple Sclerosis (MS; Encephalomyelitis disseminata; Charcot's Disease)
Myasthenia gravis (Myasthenia; MG)
Ophthalmia, sympathetic
Pemphigoid, cicatricial
Pemphigoid, bullous
Pemphigus vulgaris
Pernicious Anemia (Biermer's Disease; Addison-Anemia; see also Anemia, pernicious)
Polyarteriitis nodosa (Periarteriitis nodosa) Polychondritis (Panchondritis)
Polyglandular Autoimmune-(PGA)-Syndrome (Schmidt's Syndrome)
Polymyalgia rheumatic
Polymyositis
Polyneuropathy, Chronic-Inflammatory, Demyelinating (see also: Chronic-Inflammatory,
Demyelinating Polyneuropathy, CIDP)
Primary biliary Cirrhosis (PBC; primary Autoimmune-Cholangitis)
Psoriasis (Psoriasis)
Reiter's Syndrome (Reiter's Disease; Urethro-Conjunctival-Synovial Syndrome)
Rheumatic Fever
Great cell arteritis (see also: Temporal arteritis) Rheumatoid Arthritis (chronic Polyarthritis, “articular rheumatism”
Sarcoidosis (Boeck's Disease, Besnier-Boeck-Schaumann's Disease)
Sjögren-Syndrome
Scleroderma
Spondylitis ankylosans (see also: Bechterew's Disease)
Sprue/Celiac
Stiff-Man-Syndrome (SMS; Moersch-Woltmann-Syndrome)
Systemic Lupus erythematosus (SLE)
Takayasu Arteritis (Takayasu Disease; Aortic Arch Syndrome) Transient Gluten Intolerance
Uveitis, autoimmune
Vasculitis
Vitiligo (Vitiligo)
Wegener's Granulomatosis (see: Wegener's Disease)

4. A method according to claim 1, characterized in that the treatment covers transplant rejection.

5. A method according to claim 1, characterized in that autoantigens are obtainable by means of analytical methods.

6. A method for the identification and characterization of at least one autoantigen according to claim 1, consisting of the following steps:
a.) incubating a first array containing proteins with patient serums, blood, plasma,
b.) identifying the proteins with autoantigen-autoantibody interactions and c.) re-arraying the identified proteins into a second array, d.) incubating the array from c.) with patient serums, blood, plasma.

7. A method according to claim 6, characterized in that the array in a.) from a cDNA expression library consists of preferably more than 10,000 clones and/or proteins and contains if necessary redundant clones and/or proteins.

8. A method according to claim 1, characterized in that the support material consists of glass, carbohydrates, modified carbohydrates, sepharose, silica or organic matrices, polymers such as polyamides, PVDF, nylon, nitrocellulose, in particular available in the form of spherical, unaggregated particles such as beads, fibers or a membrane.
